SEQUENCE LISTING (2 sequences)

SEQ ID NO: 1 - protein, 7 aa, Artificial
  Synthetic minCID
TCRKHRK

SEQ ID NO: 2 - protein, 36 aa, Artificial
  Synthetic CID
KRKEVQKTCRKHRKENQGSHESPTLNPETVAINLSD

(19) United States
(12) Patent Application Publication
(10) Pub. No.: US 2019/0350907 A1
(43) Pub. Date: Nov. 21, 2019

(54) METHODS AND PHARMACEUTICAL COMPOSITIONS FOR REDUCING CD95- MEDIATED CELL MOTILITY

(71) Applicants: INSERM (INSTITUT NATIONAL DE LA SANTÉ ET DE LA RECHERCHE MÉDICALE), Paris (FR); UNIVERSITÉ DE RENNES 1, Rennes (FR); UNIVERSITÉ DE BORDEAUX, BORDEAUX (FR); INSTITUT BERGONIÉ, Bordeaux (FR); CENTRE NATIONAL DE LA RECHERCHE SCIENTIFIQUE (CNRS), Paris (FR); CHU DE BORDEAUX, Talence (FR)

(72) Inventors: Patrick LEGEMBRE, RENNES (FR); Pierre VACHER, Bordeaux cedex (FR); Amanda POISSONNIER, Rennes (FR); Patrick BLANCO, Bordeaux (FR)

(21) Appl. No.: 16/478,520

(22) PCT Filed: Jan. 15, 2018
(86) PCT No.: PCT/EP2018/050820

§ 371 Date: Jul. 17, 2019

(30) Foreign Application Priority Data
Jan. 16, 2017 (EP) 17305047.7
Jun. 6, 2017 (EP) 17305665.6
Oct. 20, 2017 (EP) 17306432.0

(51) Int. Cl. A61K 31/427; A61K 31/603; A61K 31/085; A61K 31/4439; A61K 31/704

(52) U.S. Cl. CPC A61K 31/427; A61K 31/603; A61K 31/704; A61K 31/4439; A61K 31/085

(57) ABSTRACT

The present invention relates to a method for reducing CD95-mediated cell motility. To identify chemicals disrupting CD95/PLCγ1 interaction, the inventors screened a chemical library of EMA/FDA-approved molecules against a protein-fragment complementation assay (PCA) monitoring the binding of CD95 to PLCγ1. From this screen, five chemical molecules showed the ability to disrupt CD95/PLCγ1 interaction and to neutralize the CD95-mediated calcium signaling pathway and cell migration in human peripheral blood lymphocytes (PBLs) and Th17 cells. Thus, the present invention relates to a method for reducing CD95-mediated cell motility, comprising administering the subject with at least one compound selected from the group consisting of HIV-protease inhibitors (e.g. ritonavir), diflunisal, anethole, rosiglitazone and daunorubicin. Particularly, the method of the invention find use in the treatment of cancer such as triple negative breast cancer, autoimmune inflammatory disease such as systemic lupus erythematosus, inflammatory condition and Th17-mediated disease.

### FIELD OF THE INVENTION

The present invention relates to methods and pharmaceutical compositions for reducing CD95-mediated cell motility.

### BACKGROUND OF THE INVENTION

Chronic inflammatory diseases (IDs) are the third cause of death in developed countries, after cancer and cardiovascular disorders, and their prevalence is growing in developed countries. These diseases constitute a heterogeneous group of illnesses, including autoimmune systemic diseases (systemic lupus erythematosus, systemic sclerosis (SSc)) and inflammatory bowel disorders. All these diseases appear clinically different, but in fact share many similarities, from common genetic background, common pathophysiological pathways, and not surprisingly similar treatments. From a pathogenic point of view, these IDs are usually characterized first by an autoimmune response, characterized by a breakdown of tolerance and the presence of circulating autoantibodies. Those antibodies are secreted by B cells, which have been activated by a specific subset of effector CD4+ T cells, follicular helper T cells (Tfh). Second, tissue lesions responsible for the clinical presentations involve the IL17-secreting T cells (Th17), which once recruited in the organs trigger robust inflammation. Therefore, understanding the signals governing the fate of effector T cells is of tremendous importance to identify new therapeutic targets and small molecules that interact selectively them.

Systemic Lupus Erythematosus (SLE) is a chronic auto-immune disease characterized by a loss of tolerance toward nuclear components leading to autoantibody production, immune complex formation and organ/tissue damage. Human studies and murine models indicate a role for Th17, and Tfh in SLE progression (1).

CD95L (FasL) belongs to the Tumor Necrosis Factor (TNF) family and is the ligand of the death receptor CD95 (also known as Fas). While CD95 is ubiquitously expressed on healthy cells, CD95L exhibits a restricted expression pattern, mainly detected at the surface of lymphocytes, where it plays a pivotal role in the elimination of infected and transformed cells (2). CD95L is a transmembrane glycoprotein that acts locally through cell-to-cell contact 3 and after cleavage by metalloproteases such as MMP3 (4), MMP7 (5), MMP9 (6) or A Disintegrin And Metalloproteinase 10 (ADAM-10) (7, 8), a soluble CD95L (cleaved CD95L or cl-CD95L) is released into the bloodstream. This soluble ligand contributes to aggravate inflammation in chronic inflammatory disorders such as systemic lupus erythematosus (SLE) (9, 10) by inducing non-apoptotic signaling pathways such as NF-κB (11) and PI3K (10) and may exert pro-oncogenic functions by promoting the survival of ovarian and liver cancers and chemotherapy resistance of lung cancers. CD95L receptor, designated CD95 or Fas carries an intracellular conserved stretch, the death domain (DD), which serves as a docking platform to trigger cell death. Binding of membrane-bound hexameric CD95L to CD95 leads to the recruitment of the adaptor protein FADD (Fas Associated Death Domain) through homotypic interactions via their respective DD (12). FADD in turn aggregates the initiator caspase-8 and caspase-10. The CD95/FADD/caspase complex is called death-inducing signalling complex (DISC) and leads to the elimination of cancer cells through an apoptotic mechanism (13). By contrast, homotrimeric cl-CD95L fails to induce DISC formation, but instead triggers the formation of a non-apoptotic complex termed motility-inducing signaling complex (MISC) implementing a Ca²⁺ response (10, 14, 15). Recent data from our group highlighted that cl-CD95L induces a calcium response by inducing the direct interaction of CD95 with PLCγ1 (16). Indeed, in presence of cl-CD95L, the juxtamembrane region of CD95, called calcium-inducing domain (CID), recruits PLCγ1 to induce endothelial transmigration of Th17 cells in SLE (16). Moreover, a peptide consisting of the CID conjugated to the cell-penetrating domain of TAT, namely TAT-CID, binds PLCγ1 and prevents its recruitment to CD95. Strikingly, repeated injections of TAT-CID in lupus-prone mice (MRL^{Lpr/+}) block endothelial transmigration of Th17 cells and alleviate clinical symptoms (16).

Accordingly, compounds that are able to reduce CD95-meditated cell motility are highly desirable.

### SUMMARY OF THE INVENTION

The present invention relates to methods and pharmaceutical compositions for reducing CD95-mediated cell motility. In particular, the present invention is defined by the claims.

### DETAILED DESCRIPTION OF THE INVENTION

Although CD95 ligand (CD95L) is able to trigger apoptotic cell death, its cleavage by metalloprotease generates a soluble CD95L (cl-CD95L for cleaved CD95L) failing to trigger apoptosis but inducing non apoptotic signaling pathways promoting trafficking of T helper 17 (Th17) lymphocyte in inflamed organs in lupus patients. T cell migration occurs through a direct interaction between the CD95 domain called calcium-inducing domain (CID) and the Src homology 3 (SH3) domain of phospholipase Cγ1 (Poissonnier A et al., Immunity, 2016). To identify chemicals disrupting CD95/PLCγ1 interaction, the inventors screened a chemical library of EMA/FDA-approved molecules against a protein-fragment complementation assay (PCA) monitoring the binding of CD95 to PLCγ1. From this screen, five molecules not only showed the ability to disrupt CD95/PLCγ1 interaction but also to neutralize the CD95-mediated calcium signaling pathway and cell migration. Treatment of lupus-prone mice with these molecules should alleviate the clinical symptoms and thereby, turned out to be attractive therapeutic approach.

Accordingly, one aspect of the invention relates to a method for reducing CD95-mediated cell motility in a subject in need thereof, comprising administering the subject with a therapeutically effective amount of at least one compound selected from the group consisting of HIV-protease inhibitors, diflunisal, anethole, rosiglitazone and daunorubicin.

As used herein, the term “HIV-protease inhibitor” refers to inhibitors of the HIV-1 protease, an enzyme required for the proteolytic cleavage of viral polyprotein precursors (e.g. viral GAG and GAG Pol polyproteins), into the individual functional proteins found in infectious HIV-1.

In some embodiments, the HIV-protease inhibitor of the present invention is selected from the group consisting of lopinavir, ritonavir, nelfinavir, indinavir, amprenavir, fosamprenavir, atazanavir, tipranavir, darunavir and saquinavir.

In some embodiments, the HIV-protease inhibitor of the present invention is ritonavir. As used herein, the term “ritonavir” has its general meaning in the art and refers to 1,3-thiazol-5-ylmethyl N-[(2S,3S,5S)-3-hydroxy-5-[[(2S)-3-methyl-2-[[methyl-[(2-propan-2-yl-1,3-thiazol-4-yl)methyl]carbamoyl]amino]butanoyl]amino]-1,6-diphenylhexan-2-yl]carbamate, having the molecular formula C₃₇H₄₈N₆O₅S₂ and accessible under the CAS registry number 155213-67-5. The term “ritonavir” also refers to compound described in U.S. Pat. Nos. 5,541,206; 7,364,752.

As used herein, the term “diflunisal” has its general meaning in the art and refers to 5-(2,4-difluorophenyl)-2-hydroxybenzoic acid, having the molecular formula C₁₃H₈F₂O₃ and accessible under the CAS registry number 22494-42-4. The term “diflunisal” also refers to compound described in U.S. Pat. No. 3,714,226.

As used herein, the term “anethole” has its general meaning in the art and refers to anethole trithione (5-(4-methoxyphenyl)dithiole-3-thione), having the molecular formula C₁₀H₈OS₃ and accessible under the CAS registry number CAS 532-11-6.

As used herein, the term “rosiglitazone” has its general meaning in the art and refers to 5-[[4-[2-[methyl(pyridin-2-yl)amino]ethoxy]phenyl]methyl]-1,3-thiazolidine-2,4-dione, having the molecular formula C₁₈H₁₉N₃O₃S and accessible under the CAS registry number 122320-73-4. The term “rosiglitazone” also refers to compound described in Wolffenbuttel et al., 2001; U.S. Patent US20040242658; U.S. Pat. Nos. 5,002,953; 5,965,584.

As used herein, the term “daunorubicin” has its general meaning in the art and refers to (7S,9S)-9-acetyl-7-[(2R,4S,5S,6S)-4-amino-5-hydroxy-6-methyloxan-2-yl]oxy-6,9,11-trihydroxy-4-methoxy-8,10-dihydro-7H-tetracene-5,12-dione, having the molecular formula C₂₇H₂₉NO₁₀ and accessible under the CAS registry number 20830-81-3. The term “daunorubicin” also refers to compound described in U.S. Pat. No. 3,989,598.

In some embodiments, the compounds of the present invention (e.g. a HIV protease inhibitor such as ritonavir) are particularly for reducing CD95-mediated cancer cell motility. In some embodiments, the compounds of the present invention (e.g. a HIV protease inhibitor such as ritonavir) are particularly for the treatment of cancer in a subject in need thereof.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may be treated by methods and compositions of the present invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject suffers from a cancer selected from the group consisting of breast cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers.

In some embodiments, the compounds of the present invention (e.g. a HIV protease inhibitor such as ritonavir) are particularly for the treatment of triple negative breast cancer. As used herein the expression “Triple negative breast cancer” has its general meaning in the art and means that said breast cancer lacks or expresses low levels of receptors for the hormones estrogen (ER-negative) and progesterone (PR-negative), and for the protein HER2.

In some embodiments, the compounds of the present invention (e.g. a HIV protease inhibitor such as ritonavir) are particularly for the prevention of metastases (e.g. in a subject suffering from a triple negative breast cancer).

In some embodiments, the compounds of the present invention (e.g. a HIV protease inhibitor such as ritonavir) are particularly for enhancing therapeutic efficacy of cancer treatment in a subject in need thereof. In some embodiments, the method of the present invention comprises administering the subject with a therapeutically effective amount of at least one compound selected from the group consisting of ritonavir, diflunisal, anethole, rosiglitazone and daunorubicin sequentially or concomitantly with one or more therapeutic active agent such as chemotherapeutic or radiotherapeutic agents. Examples of chemotherapeutics include but are not limited to fludarabine, gemcitabine, capecitabine, methotrexate, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbazine, epipodophyllotoxins such as etoposide and teniposide, camptothecins such as irinotecan and topotecan, bleomycin, doxorubicin, idarubicin, dactinomycin, plicamycin, mitoxantrone, L-asparaginase, doxorubicin, epirubicin, 5-fluorouracil and 5-fluorouracil combined with leucovorin, taxanes such as docetaxel and paclitaxel, levamisole, estramustine, nitrogen mustards, nitrosoureas such as carmustine and lomustine, vinca alkaloids such as vinblastine, vincristine, vindesine and vinorelbine, imatinib mesylate, hexamethylmelamine, kinase inhibitors, phosphatase inhibitors, ATPase inhibitors, tyrphostins, protease inhibitors, inhibitors herbimycin A, genistein, erbstatin, and lavendustin A. In some embodiments, additional therapeutic active agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxins, hormonal therapies, retinoids, photosensitizers or photodynamic therapies, angiogenesis inhibitors, antimitotic agents, isoprenylation inhibitors, cell cycle inhibitors, actinomycin, bleomycin, anthracyclines, MDR inhibitors and Ca2+ ATPase inhibitors. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

In some embodiments, the compounds of the present invention (e.g. a HIV protease inhibitor such as ritonavir) are particularly for reducing CD95-mediated lymphocyte (e.g., T cell) motility.

In some embodiments, the compound of the present invention (e.g. a HIV protease inhibitor such as ritonavir) are particularly suitable for inhibiting the B-cell maturation and thus the antibody production. As used herein, the term “B-cell” refers to lymphocytes that are capable of producing antibodies. These cells are the primary cell type involved in humoral acquired immunity.

In some embodiments, the compounds of the present invention (e.g. a HIV protease inhibitor such as ritonavir) are particularly for the treatment of an autoimmune inflammatory disease. In some embodiments, the autoimmune inflammatory diseases selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis (e.g. chronic pancreatitis), polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In some embodiments, the compounds of the present invention (e.g. a HIV protease inhibitor such as ritonavir) are particularly suitable for the treatment of systemic lupus erythematosus.

In some embodiments, the compounds of the present invention (e.g. a HIV protease inhibitor such as ritonavir) are particularly for the treatment of antibody-mediated diseases, including but not limited to graft rejection, graft vs. host disease, and inflammatory-autoimmune diseases (as described above). In addition, the compounds of the present invention (e.g. a HIV protease inhibitor such as ritonavir) are particularly for the treatment B-cell tumors, such as multiple myeloma and chronic lymphocytic leukemia.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

Typically, the compound of the present invention is administered to the subject in a therapeutically effective amount. By a “therapeutically effective amount” is meant a sufficient amount of the compound of the present invention for reaching a therapeutic effect (e.g. treating cancer). It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the compound of the present invention is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the compound at the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention will be further illustrated by the following figures and examples. However, these examples and figures should not be interpreted in any way as limiting the scope of the present invention.

### FIGURES

FIG. 1. Identification of chemical leads from the Prestwick library inhibiting the CD95/PLCγ1 interaction. A. Schematic representation of the protein-fragment complementation assay used with CD95-CID or -DD fused to F2 and PLCγ1-SH3 or FADD-DD fused to F1 to identify inhibitors from the Prestwick library. The Renilla luciferase enzyme was divided into amino-terminal and carboxy-terminal fragments (yellow domains) and fused to the indicated domains. B. Compounds from the Prestwick library were tested against CD95/PLCγ1 or CD95/FADD PCA. This library allowed us to select 34 hits based on their ability to inhibit CD95/PLCγ1 interaction (≥70% of signal inhibition) without altering CD95/FADD binding (ratio (CD95/PLCγ1 inhibition/CD95/FADD inhibition) ≥2). C-D. The curve (C) and the IC50 (D) of the most efficient Prestwick molecules inhibiting CD95/PLCγ1 are depicted. Inhibition of CD95/FADD interaction is also assessed.

FIG. 2. Cytotoxic effect of the Prestwick molecules on T cells. The cytotoxic effect of the indicated drugs was assessed. Indicated cells were treated for 24 hours with the indicated molecules at different concentrations and cell death was quantified using viability MTT assay as previously described (Legembre et al., Cell Death and Diff, 2002).

FIG. 3. Drugs inhibit CD95-mediated Ca′ response and cell migration. A. Activated PBLs were loaded with FuraPE3-AM (1 μM) and pre-treated for 1 h with non-toxic concentrations of indicated compounds. T-cells were then stimulated with cl-CD95L (100 ng/mL; arrow). Ratio values (R) were normalized to pre-stimulated values (R0) to yield R/R0 values. Data represent means±the SD. B. Left panel: using Boyden chamber assay, cell migration of parental H9 cells and their counterpart lacking CD95 (CD95-KO) was assessed. Right panel: activated PBLs were pre-incubated in the presence or absence of the indicated drugs for 1 hour using non-toxic concentration and the CD95-mediated cell migration was assessed by Boyden chambers. C. Th17 cells were sorted from blood of healthy donors (peripheral blood mononuclear cell). FACS analyses showing the efficiency of Th17 (CCR6⁺ CXCR3⁻) cell sorting from healthy donors PBMCs. D. Th17 cells were pre-incubated in the presence or absence of the indicated drugs for 1 hour using non-toxic concentration and cell migration was assessed by Boyden chambers.

FIG. 4. Prestwick drugs do not alter the ER calcium store.

A. Activated PBLs were loaded with FuraPE3-AM (1 μM) and pre-treated for 1 h with non-toxic concentrations of indicated compounds. T-cells were then stimulated with thapsigargin (1 μM; arrow). Ratio values (R) were normalized to pre-stimulated values (R0) to yield R/R0 values. Data represent means±the SD. B. Th17 cells were sorted from healthy donors and loaded with FuraPE3-AM (1 μM). Th17 cells were pre-treated for 1 h with non-toxic concentrations of ritonavir or diflunisal (1 μM). T-cells were then stimulated with cl-CD95L (100 ng/mL; arrow). Ratio values (R) were normalized to pre-stimulated values (R0) to yield R/R0 values. Data represent means±the SD.

FIG. 5. CID and minCID inhibit CD95/PLCγ1 interaction.

A. CID-CD95 (aa 175-210) or DD-CD95 (aa 210-303) fused to F2 were co-transfected into HEK cells with the indicated domains of PLCγ1 (Ca²⁺ response) or FADD (apoptosis) fused to F1. Light emission indicates refolding of the luciferase and reconstitution of enzyme activity through protein/protein interactions. The inhibition of light emission for the indicated PPIs was assessed in cells incubated for the indicated times in the presence of TAT-CID (25 μM). Data represent means±SD of three independent experiments. B. The minimal domain of CID responsible for PLCγ1 binding is depicted (SEQ ID NO:2). C-D. The PLCγ1-SH3 domain (aa 790-851) (C) and the death domain of FADD (aa 80-210) (D) fused to F1 was co-transfected into HEK cells with the whole CID-CD95 or DD-CD95 fused to F2. Then, the indicated peptides fused with or without the cell-penetrating domain TAT were incubated at the indicated concentrations for 4 hrs and luminescence was assessed. Data represent means±SD of three independent experiments. Data represent means±the SD of 3 independent experiments.

FIG. 6: CI-CD95L induces the endothelial transmigration of Tfh cells. Tfh cells were pre-treated in the presence or absence of TAT-ctr, TAT-CID or ritonavir (1 μM) for 1 hr and then stimulated with or without cl-CD95L (100 ng/mL). Endothelial transmigration of Tfh cells was assessed by measuring their passage across the porous membrane of a Boyden chamber that has been covered by a monolayer of endothelial cells (HUVEC).

FIG. 7: HIV protease inhibitors inhibit the CD95-mediated Ca²⁺ signaling pathway in Th17 and Tfh cells. Human PBLs were sorted using flow cytometry cell sorting to isolate Th17 (A) according to their phenotype (CD3+CD4+CXCR5−CCR6+CXCR3−CD45RA−) and Tfh (B) (CD4+CXCR5+ICOS+PD−1+CD45RA−). Cells were pre-incubated with Indicated drugs (1 μM) for 1 h and then stimulated with 100 ng/mL of s-CD95L. [Ca2+]CYT was assessed in Fluo2 LR-AM (3 μM)-loaded cells. Data represent means±the SD of 3 independent experiments.

### EXAMPLE 1

Material & Methods

In Vitro Motility Assays.

After membrane hydration of Boyden chambers (Millipore, Molsheim, France) containing 8 μm pore membranes, 10⁵ cells were added to the top chamber. The bottom chamber was filled with low serum (1%)-containing medium in the presence or absence of cl-CD95L (100 ng/ml). Cells were incubated for 24 h. To quantify invasion, cells were fixed with methanol and stained with Giemsa. Stained cells were then removed from the top-side of the membrane using a cotton-tipped swab and five representative pictures for each insert were taken of the invading cells from the reverse side. For each experiment, invading cells were lysed and absorbance at 560 nm was measured.

Cell Lines and Peripheral Blood Lymphocytes

All cells were purchased from ATCC (Molsheim Cedex, France). The T-cell lines H9, CEM and H9 cell lacking CD95 (CRISPR/cas9, Horizon, Cambridge, United Kingdom) were cultured in RPMI supplemented with 8% heat-inactivated FCS (v/v) and 2 mM L-glutamine at 37° C. in a 5% CO2 incubator. HEK293 cells were cultured in DMEM supplemented with 8% heat-inactivated FCS and 2 mM L-glutamine at 37° C. in a 5% CO₂ incubator. PBMCs (peripheral blood mononuclear cells) from healthy donors were isolated by Ficoll centrifugation, washed twice in PBS. Monocytes were removed by a 2 hours adherence step and the naive PBLs (peripheral blood lymphocytes) were incubated overnight in RPMI supplemented with 1 μg/ml of PHA. Cells were washed extensively and incubated in the culture medium supplemented with 100 units/ml of recombinant IL-2 (R&D) for 5 days. Human umbilical vein endothelial cell (HUVEC) (19) were grown in human endothelial serum free medium 200 supplemented with LSGS (Low serum growth supplement) (Invitrogen, Cergy Pontoise, France).

Renilla Luciferase-Based Protein Fragment Complementation Assay

HEK293T cells were electroporated with 5 μg of DNA using BTM-830 electroporation generator (BTX Instrument Division, Harvard Apparatus) with 10 μg of DNA. Transfected cells were cultured for 24 hrs prior to protein complementation assay analyses as previously described (17). Briefly, transfected cells (10⁶) were washed, resuspended in 100 μl PBS and deposited in OptiPlate-96 (PerkinElmer, Waltham, Mass., USA). Coelenterazine-h (5 μM, Sigma) was injected to each well and renilla luciferase activity was monitored for the first 10 seconds using Infinite200Pro (Tecan, Männedorf, Switzerland).

T Cell Subset Isolation (Th17)

Peripheral blood mononuclear cells (PBMC) were isolated from buffy-coat by density gradient using lymphocyte separation medium (Eurobio, Les Ulis, France). PBMCs were then subjected to selection using a cocktail of antibody-coated magnetic beads: CCR6⁺ CXCR3⁻CD4⁺ cells (Th17 cells) were sorted with Human Th17 Enrichment kit (STEMCELL Technologies, Grenoble, France). Cell sorting efficiency was evaluated by FACS.

Transendothelial Migration of Activated T Lymphocytes

Membranes (3 μm pore size) of a Boyden chamber were hydrated in sterile D-PBS (Millipore, Molsheim, France). Thereafter, activated T-lymphocytes (10⁶) were added to the top chamber on a confluent monolayer of HUVEC in a low serum (1%)-containing medium. The bottom chamber was filled with low serum (1%)-containing medium in presence or absence of 100 ng/ml of cl-CD95L. In experiments using human sera, 500 μl of serum from either healthy donors or SLE patients was added to the lower chamber. Cells were cultured for 24 h at 37° C. in a 5% CO2, humidified incubator. Transmigrated cells were counted in the lower reservoir by flow cytometry using a standard of 2.5×10⁴ fluorescent beads (Flow-count, Beckman Coulter).

Video Imaging of the Calcium Response in Living Cells

T-cells were loaded with Fura-PE3-AM (1 μM) at room temperature for 30 min in Hank's Balanced Salt Solution (HBSS). After washing, the cells were incubated for 15 min in the absence of Fura-PE3-AM to complete de-esterification of the dye. Cells were placed in a temperature controlled chamber (37° C.) of an inverted epifluorescence microscope (Olympus IX70) equipped with an x40 UApo/340-1.15 W water-immersion objective (Olympus), and fluorescence micrograph images were captured at 510 nm and 12-bit resolution by a fast-scan camera (CoolSNAP fx Monochrome, Photometrics). To minimize UV light exposure, a 4×4 binning function was used. Fura-PE3 was alternately excited at 340 and 380 nm, and the ratios of the resulting images (emission filter at 520 nm) were produced at constant intervals (10 seconds). The Fura-PE3 ratio (Fᵣₐₜᵢₒ 340/380) images were analyzed offline with Universal Imaging software, including Metafluor and Metamorph. Fᵣₐₜᵢₒ reflects the intracellular Ca²⁺ concentration changes. Each experiment was repeated at least 3 times, and the average of more than 20 single-cell traces per experiment was analyzed.

Results

Identification of Compounds Inhibiting CD95/PLCγ1 Interaction.

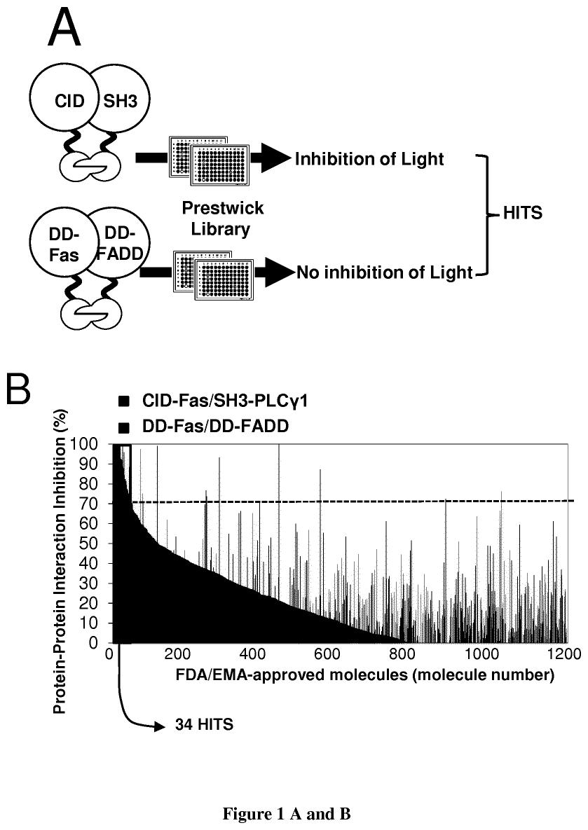

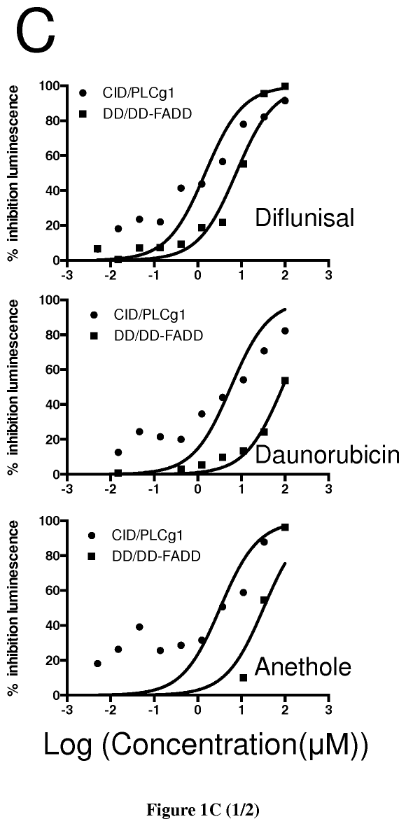

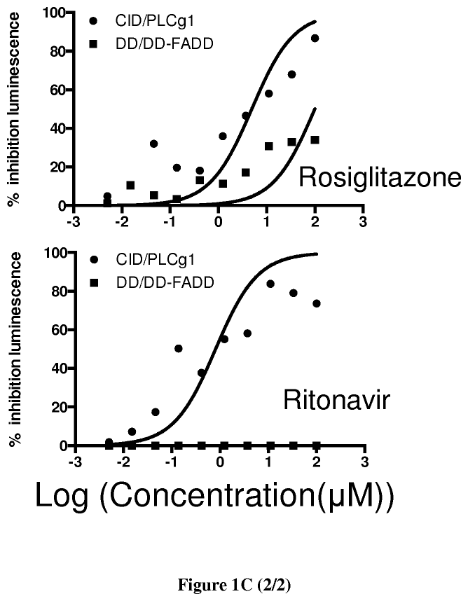

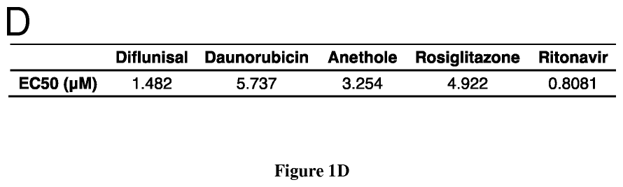

We recently demonstrated that amino acid residues 175 to 210 of CD95 designated calcium-inducing domain (CID) bind PLCγ1 to induce a calcium response promoting metastasis of cancer cells in triple negative breast cancer (TNBC) patients (15) and accumulation of CD4⁺ Th17 cells in damaged organs of systemic lupus erythematosus (SLE) patients (10). A peptide consisting of the cell-penetrating domain of TAT fused to CID (TAT-CID) binds the SH3 domain of PLCγ1 and thereby, prevents PLCγ1/CD95 interaction and the subsequent induction of the Ca²⁺ response (16). Although TAT-CID impedes the CD95-mediated Ca²⁺ response, it does not affect the apoptotic signaling pathway; TAT-CID represents the first member of a novel generation of selective inhibitors with therapeutic potential to dampen inflammation in auto-immune disorders (16) and prevent metastasis in TNBC patients (15) without affecting the pro-apoptotic and anti-tumor role of CD95. Accordingly, we developed an assay designated protein-fragment complementation assay (PCA) to perform high-throughput screening and identify a new generation of CD95/PLCγ1 interaction inhibitors that do not affect CD95/FADD binding. Briefly, the Renilla luciferase enzyme was divided into two fragments (F1 and F2 fragments). F2 fragment was fused to CID (i.e., amino acids 175 to 210) or DD (i.e., amino acids 210 to 303) of CD95, while F1 was associated with SH3-PLCγ1 or DD-FADD and these F1 and F2 domains were co-expressed in HEK cells in which the functional reconstitution of RLuc was assessed (17). Interaction of CD95 with PLCγ1 or FADD allows the reconstitution of RLuc and subsequent light emission (FIG. 1A). The chemical Prestwick library was screened against these PCAs (FIG. 1B). This library encompasses a collection of 1280 small molecules, which are FDA and/or EMA approved and have been selected for their high structural diversity, bioavailability and safety in humans. Therefore, they represent the greatest level of druglikeness. The screen revealed 34 hits selectively abrogating CD95/PLCγ1 interaction without altering CD95/FADD binding (inhibition superior to 70% of the maximal luminescent signal obtained without the drug and ratio of CD95/PLCγ1/CD95/FADD inhibition ≥2). IC₅₀ analyses with these 34 hits finally revealed that 5 molecules only inhibited CD95/PLCγ1 interaction with an IC₅₀ lower than 10 μM (FIG. 1C-D). These molecules are designated CID inhibitors or CIDINH.

CID Inhibitors Abrogate the CD95-Mediated Ca²⁺ Response and Cell Migration.

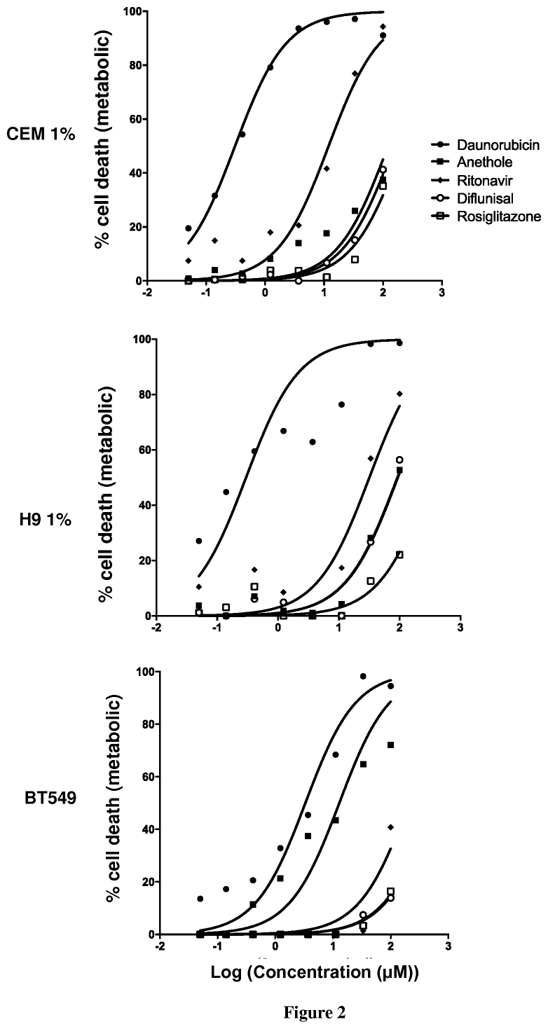

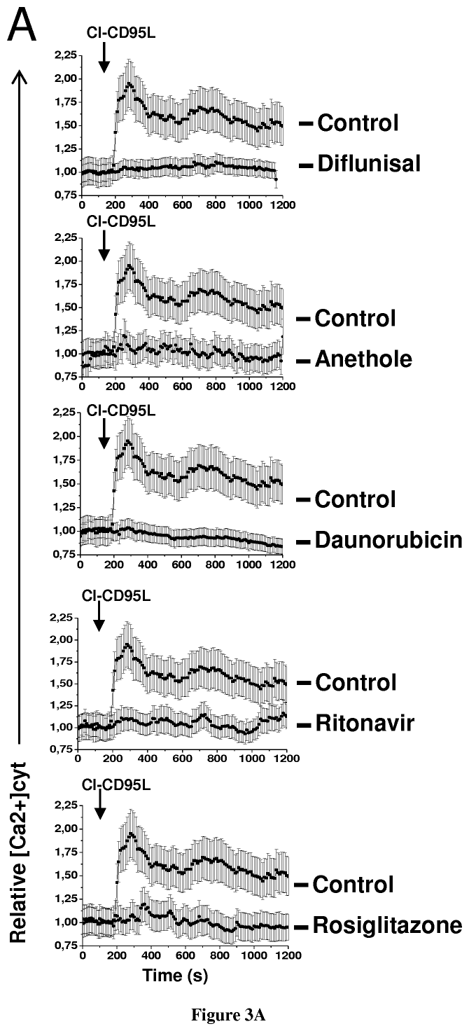

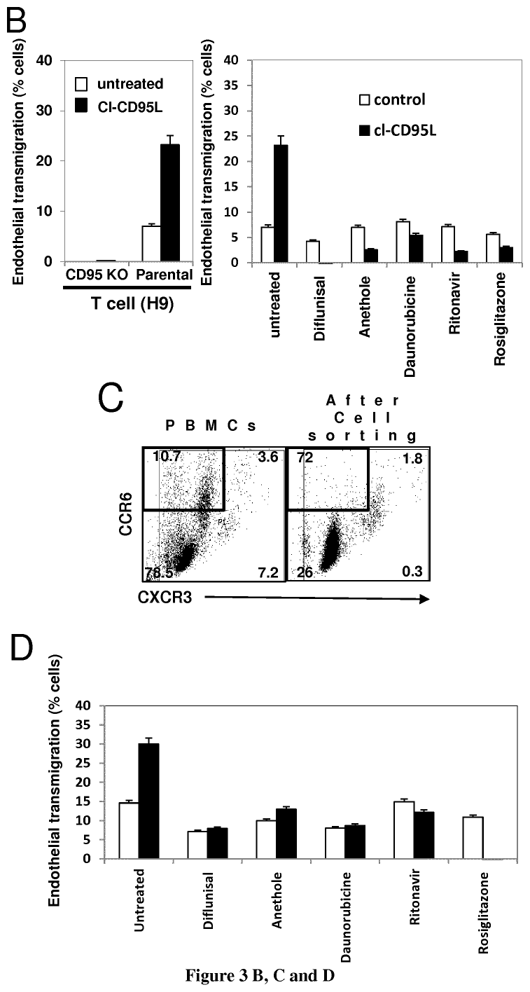

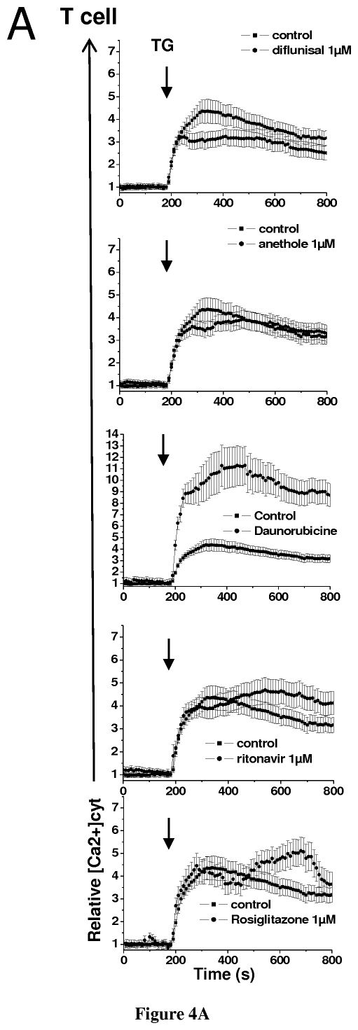

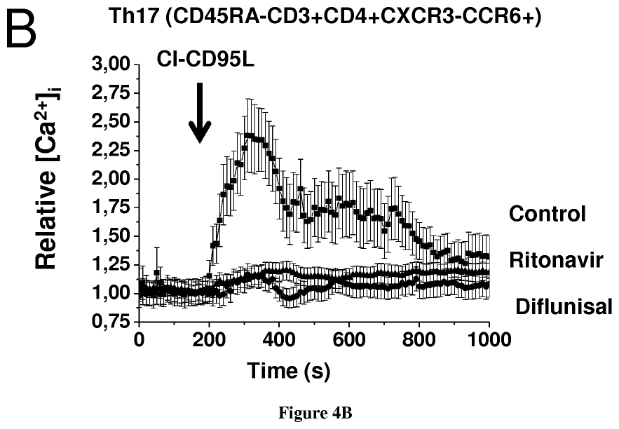

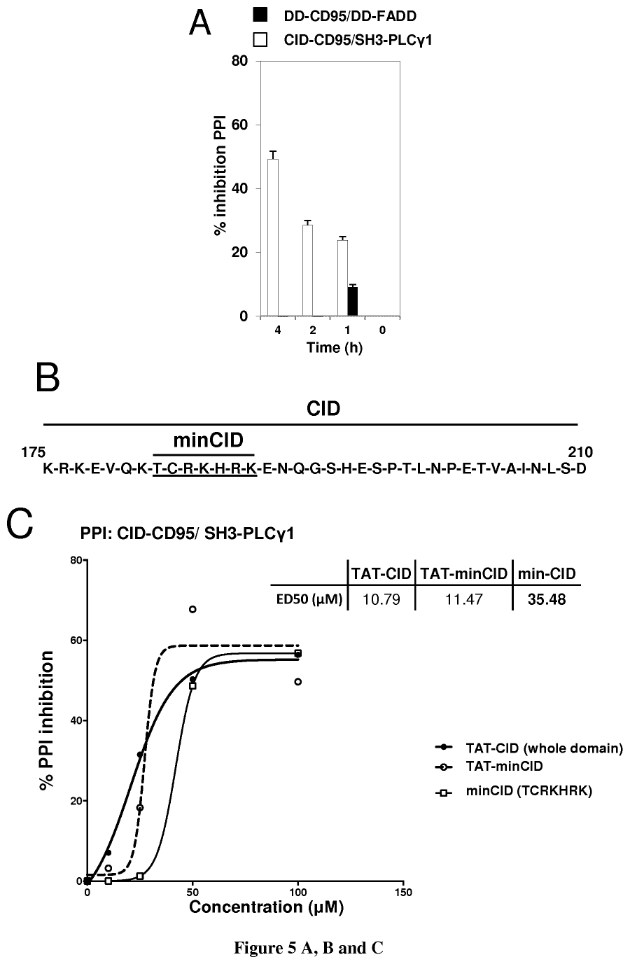

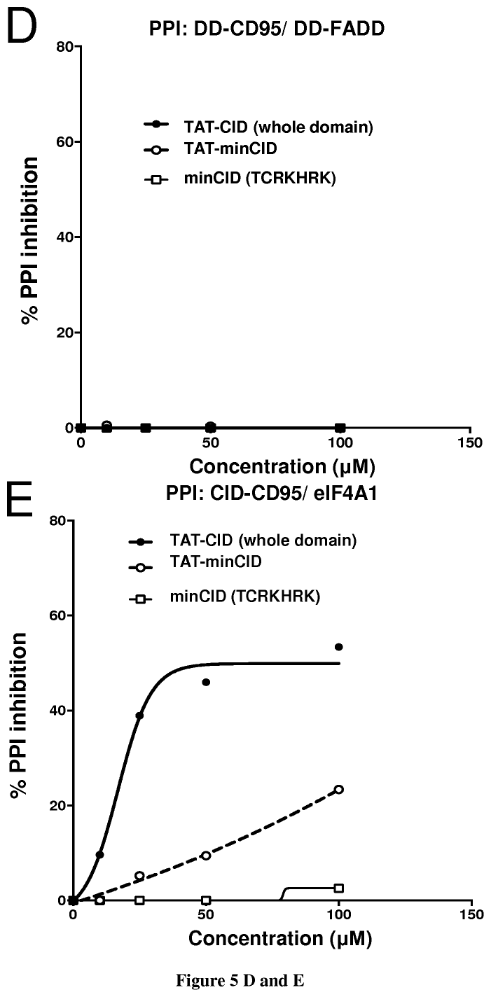

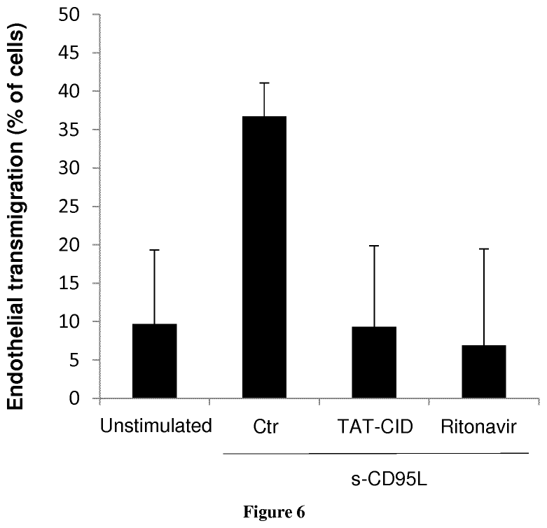

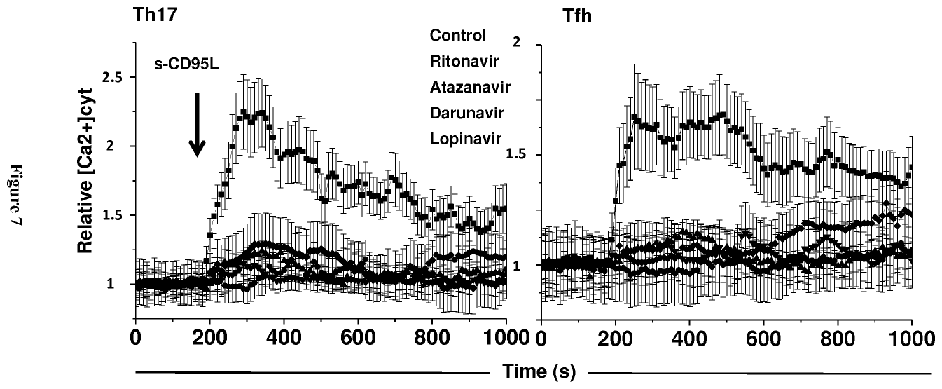

We next wondered whether CIDINH were able to inhibit CD95-mediated Ca²⁺ signaling pathway (FIG. 3A) and cell migration (FIG. 3B) in activated PBLs at non-toxic concentrations (FIG. 2). Strikingly, similar data were obtained with CCR6⁺ CXCR3⁻ Th17 cells isolated from healthy donors (FIG. 3 C-D). Of note, inhibition of the CD95-mediated Ca²⁺ response was not due to a depletion of Ca²⁺ from the endoplasmic reticulum (ER) because thapsigargine (TG), a non-competitive inhibitor of the sarco/endoplasmic reticulum Ca²⁺ ATPase, did not alter the ER calcium concentration in cells treated with the different drugs (FIG. 4). Overall, these findings indicated that five small molecules used in the clinic to treat different pathologies such as diabetes (rosiglitazone), lymphoma (daunorubicin) or HIV (ritonavir) and prescribed as a nonsteroidal anti-inflammatory drug (diflunisal) or choleretic (anethole) efficiently prevented the interaction of CD95 with PLCγ1 and thereby inhibited endothelial transmigration of PBLs. Selected according to their best IC₅₀, we next evaluated the effect of diflunisal and ritonavir on the CD95-mediated Ca²⁺ response in T-cells involved in lupus pathogenesis. Non-toxic concentrations of diflunisal and ritonavir abrogated the CD95-mediated Ca²⁺ signaling (FIG. 4B) and endothelial transmigration in Th17 cells (FIG. 3D) suggesting that diflunisal and ritonavir could represent attractive therapeutic molecules to treat lupus-prone mice MRL^{Lpr/+}.

The HIV Protease Inhibitor Ritonavir and Minimal CID Shares Some Common Features in their Manner they Interact with the SH3 Domain of PLCγ1

Two computational analyses estimated that amino acids 182 to 188 (TCRKHRK (SEQ ID NO:1), namely minCID for minimal CID, of CD95 had the highest affinity for PLCγ1-SH3 (16) and the calculated binding energy of the complex consisting of PLCγ1-SH3 and TCRKHRK (SEQ ID NO:1) was similar to that of PLCγ1 with its natural ligand SLP-76 (16). We then wondered whether, like the 36 amino-acid sequence of CID, the heptapeptide minCID would be able to disrupt the CD95/PLCγ1 interaction. Because inhibition of CD95/PLCγ1 by TAT-CID increased in a time dependent manner (FIG. 5A), we performed the next PCA experiments incubating cells with peptides for 4 hours. While both TAT-CID and TAT-minCID (FIG. 5B) disrupted CD95/PLCγ1 interaction with similar efficacy (IC₅₀ 10.79 vs 11.47 μM, respectively) (FIG. 5C), the peptides failed to alter CD95/FADD interaction (FIG. 5D) confirming the favorable selectivity of these molecules. Of note, the peptide minCID is short (not exceeding 30 residues) and positively charged, key features of a cell-penetrating peptide (CPPs) (18). Based on these features, we next evaluated whether minCID (lacking the TAT sequence), could exert inhibitory effect on the CD95/PLCγ1 interaction in non-permeabilized cells. Even though minCID disrupted the CD95/PLCγ1 interaction (FIG. 5C) to a lesser extent than its TAT-CID (IC₅₀ 35.48 vs 11.47 μM, FIG. 5C), this finding clearly demonstrated that this short positively charged peptide was able to cross the plasma membrane and to bind PLCγ1 and disrupt the desired PPI, highlighting that minCID corresponds to a surgical inhibitor of the CD95/PLCγ1 interaction that can help us to better understand the pathophysiological roles of the CD95-mediated calcium response. Comparative analysis of the docking model of minCID and ritonavir revealed that their modes of binding with PLCγ1-SH3 shared common features. Predicted interaction between CID and SH3 domain of PLCγ1 was performed using two molecular modeling approaches to uncover TCRKHRK (SEQ ID NO:1) as the optimal aa sequence within CD95-CID for interaction with PLCγ1-SH3. Superimposition of ritonavir and minCID reveals that these compounds share some structural features. Using dynamic modeling, we evaluated the PLCγ1-SH3 binding interactions shared by CID and ritonavir to identify a pharmacophore, allowing us to propose a new generation of CD95/PLCγ1 inhibitors (data not shown).

### REFERENCES

1. Shin, M. S.; Lee, N.; Kang, I. Effector T-cell subsets in systemic lupus erythematosus: update focusing on Th17 cells. Curr Opin Rheumatol. 2011, 23, 444-448.
2. Strasser, A.; Jost, P. J.; Nagata, S. The many roles of FAS receptor signaling in the immune system. Immunity. 2009, 30, 180-192.
3. Suda, T.; Takahashi, T.; Golstein, P.; Nagata, S. Molecular cloning and expression of the Fas ligand, a novel member of the tumor necrosis factor family. Cell. 1993, 75, 1169-1178.
4. Matsuno, H.; Yudoh, K.; Watanabe, Y.; Nakazawa, F.; Aono, H.; Kimura, T. Stromelysin-1 (MMP-3) in synovial fluid of patients with rheumatoid arthritis has potential to cleave membrane bound Fas ligand. J Rheumatol. 2001, 28, 22-28.
5. Vargo-Gogola, T.; Crawford, H. C.; Fingleton, B.; Matrisian, L. M. Identification of novel matrix metalloproteinase-7 (matrilysin) cleavage sites in murine and human Fas ligand. Arch Biochem Biophys. 2002, 408, 155-161.
6. Kiaei, M.; Kipiani, K.; Calingasan, N. Y.; Wille, E.; Chen, J.; Heissig, B.; Rafii, S.; Lorenzl, S.; Beal, M. F. Matrix metalloproteinase-9 regulates TNF-alpha and FasL expression in neuronal, glial cells and its absence extends life in a transgenic mouse model of amyotrophic lateral sclerosis. Exp Neurol. 2007, 205, 74-81.
7. Kirkin, V.; Cahuzac, N.; Guardiola-Serrano, F.; Huault, S.; Luckerath, K.; Friedmann, E.; Novac, N.; Wels, W. S.; Martoglio, B.; Hueber, A. O.; Zornig, M. The Fas ligand intracellular domain is released by ADAM10 and SPPL2a cleavage in T-cells. Cell Death Differ. 2007, 14, 1678-1687.
8. Schulte, M.; Reiss, K.; Lettau, M.; Maretzky, T.; Ludwig, A.; Hartmann, D.; de Strooper, B.; Janssen, O.; Saftig, P. ADAM10 regulates FasL cell surface expression and modulates FasL-induced cytotoxicity and activation-induced cell death. Cell Death Differ. 2007, 14, 1040-1049.
9. O'Reilly, L. A.; Tai, L.; Lee, L.; Kruse, E. A.; Grabow, S.; Fairlie, W. D.; Haynes, N. M.; Tarlinton, D. M.; Zhang, J. G.; Belz, G. T.; Smyth, M. J.; Bouillet, P.; Robb, L.; Strasser, A. Membrane-bound Fas ligand only is essential for Fas-induced apoptosis. Nature. 2009, 461, 659-663.
10. Tauzin, S.; Chaigne-Delalande, B.; Selva, E.; Khadra, N.; Daburon, S.; Contin-Bordes, C.; Blanco, P.; Le Seyec, J.; Ducret, T.; Counillon, L.; Moreau, J. F.; Hofman, P.; Vacher, P.; Legembre, P. The naturally processed CD95L elicits a c-yes/calcium/PI3K-driven cell migration pathway. PLoS Biol. 2011, 9, e1001090.
11. O'Reilly, K. E.; Rojo, F.; She, Q. B.; Solit, D.; Mills, G. B.; Smith, D.; Lane, H.; Hofmann, F.; Hicklin, D. J.; Ludwig, D. L.; Baselga, J.; Rosen, N. mTOR inhibition induces upstream receptor tyrosine kinase signaling and activates Akt. Cancer Res. 2006, 66, 1500-1508.
12. Holler, N.; Tardivel, A.; Kovacsovics-Bankowski, M.; Hertig, S.; Gaide, O.; Maranon, F.; Tinel, A.; Deperthes, D.; Calderara, S.; Schulthess, T.; Engel, J.; Schneider, P.; Tschopp, J. Two adjacent trimeric Fas ligands are required for Fas signaling and formation of a death-inducing signaling complex. Mol Cell Biol. 2003, 23, 1428-1440.
13. Kischkel, F. C.; Hellbardt, S.; Behrmann, I.; Germer, M.; Pawlita, M.; Krammer, P. H.; Peter, M. E. Cytotoxicity-dependent APO-1 (Fas/CD95)-associated proteins form a death-inducing signaling complex (DISC) with the receptor. Embo J. 1995, 14, 5579-5588.
14. Kleber, S.; Sancho-Martinez, I.; Wiestler, B.; Beisel, A.; Gieffers, C.; Hill, O.; Thiemann, M.; Mueller, W.; Sykora, J.; Kuhn, A.; Schreglmann, N.; Letellier, E.; Zuliani, C.; Klussmann, S.; Teodorczyk, M.; Grone, H. J.; Ganten, T. M.; Sultmann, H.; Tuttenberg, J.; von Deimling, A.; Regnier-Vigouroux, A.; Herold-Mende, C.; Martin-Villalba, A. Yes and PI3K bind CD95 to signal invasion of glioblastoma. Cancer Cell. 2008, 13, 235-248.
15. Malleter, M.; Tauzin, S.; Bessede, A.; Castellano, R.; Goubard, A.; Godey, F.; Leveque, J.; Jezequel, P.; Campion, L.; Campone, M.; Ducret, T.; Macgrogan, G.; Debure, L.; Collette, Y.; Vacher, P.; Legembre, P. CD95L cell surface cleavage triggers a prometastatic signaling pathway in triple-negative breast cancer. Cancer Res. 2013, 73, 6711-6721.
16. Poissonnier, A.; Sanseau, D.; Le Gallo, M.; Malleter, M.; Levoin, N.; Viel, R.; Morere, L.; Penna, A.; Blanco, P.; Dupuy, A.; Poizeau, F.; Fautrel, A.; Seneschal, J.; Jouan, F.; Ritz, J.; Forcade, E.; Rioux, N.; Contin-Bordes, C.; Ducret, T.; Vacher, A. M.; Barrow, P. A.; Flynn, R. J.; Vacher, P.; Legembre, P. CD95-Mediated Calcium Signaling Promotes T Helper 17 Trafficking to Inflamed Organs in Lupus-Prone Mice. Immunity. 2016, 45, 209-223.
17. Stefan, E.; Aquin, S.; Berger, N.; Landry, C. R.; Nyfeler, B.; Bouvier, M.; Michnick, S. W. Quantification of dynamic protein complexes using Renilla luciferase fragment complementation applied to protein kinase A activities in vivo. Proc Natl Acad Sci USA. 2007, 104, 16916-16921.
18. Bechara, C.; Sagan, S. Cell-penetrating peptides: 20 years later, where do we stand? FEBS Lett. 2013, 587, 1693-1702.
19. Jaffe, E. A.; Nachman, R. L.; Becker, C. G.; Minick, C. R. Culture of human endothelial cells derived from umbilical veins. Identification by morphologic and immunologic criteria. J Clin Invest. 1973, 52, 2745-2756.
20. Kikawada, E.; Lenda, D. M.; Kelley, V. R. IL-12 deficiency in MRL-Fas(lpr) mice delays nephritis and intrarenal IFN-gamma expression, and diminishes systemic pathology. J Immunol. 2003, 170, 3915-3925.
21. Legembre, P.; Moreau, P.; Daburon, S.; Moreau, J. F.; Taupin, J. L. Potentiation of Fas-mediated apoptosis by an engineered glycosylphosphatidylinositol-linked Fas. Cell Death Differ. 2002, 9, 329-339.

### EXAMPLE 2: RITONAVIR INHIBITS SOLUBLE CD95L-MEDIATED T-CELL DEPENDENT B-CELL MATURATION INTO LONG-LIVED PLASMA CELLS

We recently observed that sCD95L interaction with CD95 triggers a Ca²⁺ signal, which promotes endothelial transmigration of Th17 cells¹⁹. Because the amount of anti-double strand DNA antibodies dropped in lupus-prone mice in which the sCD95L signal was inhibited, we next investigated whether sCD95L may affect B cell maturation and Ig production. Although sCD95L induces a weak Ca²⁺ response in Treg cells, this signal was similar between Th17 and circulating Tfh cells. Two selective inhibitors of the CD95-mediated non-apoptotic signaling pathway, TAT-CID and ritonavir abrogated this Ca2⁺response Like Th17 cells, circulating Tfh exposed to sCD95L underwent a cellular program promoting their endothelial transmigration (FIG. 6), a cellular process crucial for their recruitment into secondary lymphoid organs in which they will induce B-cell maturation. To decipher whether s-CD95L also affected Tfh-dependent B-cell maturation, we next incubated memory B cells with Tfh cells stimulated with SEB in the presence or absence of s-CD95L. We observed that s-CD95L significantly enhanced the ability of Tfh to induce the differentiation of memory B cells into plasmablast (CD19⁺CD27⁺CD38⁺) in a dose-dependent manner (data not shown). In agreement with this improved B-cell maturation, addition of s-CD95L also enhanced IgG levels in the supernatant of the co-cultures. This observation was directly dependent of the interaction between s-CD95L and CD95 expressed by Tfh cells, because preincubation of Tfh cells with either Tat-CID or Ritonavir blocks this Tfh-dependent B cell activation. Altogether these observations unravels a new role of s-CD95L in T-cell-dependent B cell homeostasis justify the use of Ritonavir for the treatment of autoimmune diseases including SLE.

### EXAMPLE 3

Since the discovery of HIV, 26 anti-HIV drugs have been approved by the US Food and Drug Administration (FDA) and among these compounds, ten are HIV protease inhibitors. HIV protease cleaves Gag and Gag-Pol polyprotein precursors to generate mature proteins indispensable for the viral maturation, rendering this molecule very attractive as a therapeutic target. HIV protease inhibitors share structural similarities and interact with the HIV protease in a similar fashion¹. Indeed, two HIV protease subunits come together to form a catalytic tunnel interacting with nascent peptides and cleaving them².

Structure-activity relationships and crystallographic analyses have shown that very potent HIV proteases inhibitors could be obtained with rather simple design principles: a tetrahedral atom bearing hydroxyl group(s) to mimic the transition state, and bulky hydrophobic groups on each side of this central atom along the enzyme's C2 axis of symmetry.

Our High Throughput Screening (HTS) revealed that the HIV protease inhibitor ritonavir interacts with the PLCγ1 SH3 domain and prevents the PLCγ1 recruitment to the calcium-inducing domain (CID) of CD95 (see EXAMPLE 1). Comparison between the crystallized HIV protease/ritonavir and the modeled SH3/ritonavir structures indicated that in both complexes, the hot spots of interaction consist of two neighboring hydrophobic patches, lying on each side of the drug hydroxyl group: amino acid residues I50, V82, 184³ for HIV protease, or amino acids Y802, P842, Y845, W828, N844 for the PLCγ1 SH3 domain⁴. For each protein, these positions correspond to cavities hosting the benzyl substituents of ritonavir (data not shown). While the central hydroxyl group is mandatory for potent HIV protease inhibitors⁵, this function in ritonavir could impede its interaction with the hydrophobic SH3 surface. However, it appears solvent-exposed in the modeled complex, with no protein interaction. In brief, PLCγ1 SH3 binding site has certainly no clear preference at this position, and can tolerate any polar group (a ketone according to modeled CID⁴ and co-crystallized SLP76 peptides⁶, data not shown). Finally, the bound conformation of ritonavir and analogues within HIV protease is very similar to that within PLCγ1, describing a “stairstep-like” structure as observed for co-crystallized HIV protease inhibitors.

Based on the shared structural features of HIV protease inhibitors (data not shown), we further investigated whether other HIV protease inhibitors including lopinavir, atazanavir and darunavir inhibited the CD95-mediated Ca²⁺ signaling pathway. As shown in FIG. 7, all protease inhibitors efficiently abrogated the CD95-mediated Ca²⁺ signaling pathway in both Th17 and Tfh cells indicating that these drugs correspond to attractive therapeutic options for the treatment of Th17 and Tfh (B-cell)-driven pathologies including lupus.

### REFERENCES

1 Lv, Z., Chu, Y. & Wang, Y. HIV protease inhibitors: a review of molecular selectivity and toxicity. HIV AIDS (Auck1) 7, 95-104, doi:10.2147/HIV.S79956 (2015).
2 Navia, M. A. et al. Three-dimensional structure of aspartyl protease from human immunodeficiency virus HIV-1. Nature 337, 615-620, doi:10.1038/337615a0 (1989).
3 Henderson, L. E. et al. Molecular characterization of gag proteins from simian immunodeficiency virus (SIVMne). Journal of virology 62, 2587-2595 (1988).
4 Poissonnier, A. et al. CD95-Mediated Calcium Signaling Promotes T Helper 17 Trafficking to Inflamed Organs in Lupus-Prone Mice. Immunity 45, 209-223, doi:10.1016/j.immuni.2016.06.028 (2016).
5 Rich, D. H., Green, J., Toth, M. V., Marshall, G. R. & Kent, S. B. Hydroxyethylamine analogues of the p17/p24 substrate cleavage site are tight-binding inhibitors of HIV protease. Journal of medicinal chemistry 33, 1285-1288 (1990).
6 Deng, L., Velikovsky, C. A., Swaminathan, C. P., Cho, S. & Mariuzza, R. A. Structural basis for recognition of the T cell adaptor protein SLP-76 by the SH3 domain of phospholipase Cgamma1. Journal of molecular biology 352, 1-10, doi:10.1016/j.jmb.2005.06.072 (2005).
7 Erickson, J. et al. Design, activity, and 2.8 A crystal structure of a C2 symmetric inhibitor complexed to HIV-1 protease. Science 249, 527-533 (1990).

### REFERENCES

Throughout this application, various references describe the state of the art to which this invention pertains. The disclosures of these references are hereby incorporated by reference into the present disclosure.

1. A method for reducing CD95-mediated motility of a cell in a subject in need thereof, comprising administering to the subject a therapeutically effective amount of at least one compound selected from the group consisting of an HIV-protease inhibitor, diflunisal, anethole, rosiglitazone and daunorubicin.

2. The method of claim 1 wherein the HIV-protease inhibitor is selected from the group consisting of lopinavir, ritonavir, nelfinavir, indinavir, amprenavir, fosamprenavir, atazanavir, tipranavir, darunavir and saquinavir.

3. The method of claim 1 wherein the HIV-protease inhibitor is ritonavir.

4. The method of claim 1 wherein the cell is a cancer cell.

5. A method of treating cancer in a subject in need thereof, comprising administering to the subject a therapeutically effective amount of at least one compound selected from the group consisting of an HIV-protease inhibitor, diflunisal, anethole, rosiglitazone and daunorubicin.

6. The method of claim 5 wherein the cancer is selected from the group consisting of neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

7. The method of claim 5 wherein the subject suffers from a cancer selected from the group consisting of breast cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers.

8. The method of claim 5 wherein the cancer is triple negative breast cancer.

9. (canceled)

10. A method of treating an autoimmune inflammatory disease in a subject in need thereof, comprising administering to the subject a therapeutically effective amount of at least one compound selected from the group consisting of an HIV-protease inhibitor, diflunisal, anethole, rosiglitazone and daunorubicin.

11. The method of claim 10 wherein the autoimmune inflammatory disease is selected from the group consisting of Addison's Disease, Allergy, Alopecia Areata, Alzheimer's disease, Antineutrophil cytoplasmic antibodies (ANCA)-associated vasculitis, Ankylosing Spondylitis, Antiphospholipid Syndrome (Hughes Syndrome), arthritis, Asthma, Atherosclerosis, Atherosclerotic plaque, autoimmune disease (e.g., lupus, RA, MS, Graves' disease, etc.), Autoimmune Hemolytic Anemia, Autoimmune Hepatitis, Autoimmune inner ear disease, Autoimmune Lymphoproliferative syndrome, Autoimmune Myocarditis, Autoimmune Oophoritis, Autoimmune Orchitis, Azoospermia, Behcet's Disease, Berger's Disease, Bullous Pemphigoid, Cardiomyopathy, Cardiovascular disease, Celiac Sprue/Coeliac disease, Chronic Fatigue Immune Dysfunction Syndrome (CFIDS), Chronic idiopathic polyneuritis, Chronic Inflammatory Demyelinating, Polyradicalneuropathy (CIPD), Chronic relapsing polyneuropathy (Guillain-Barré syndrome), Churg-Strauss Syndrome (CSS), Cicatricial Pemphigoid, Cold Agglutinin Disease (CAD), chronic obstructive pulmonary disease (COPD), CREST syndrome, Crohn's disease, Dermatitis, Herpetiformus, Dermatomyositis, diabetes, Discoid Lupus, Eczema, Epidermolysis bullosa acquisita, Essential Mixed Cryoglobulinemia, Evan's Syndrome, Exopthalmos, Fibromyalgia, Goodpasture's Syndrome, Hashimoto's Thyroiditis, Idiopathic Pulmonary Fibrosis, Idiopathic Thrombocytopenia Purpura (ITP), IgA Nephropathy, immunoproliferative disease or disorder (e.g., psoriasis), Inflammatory bowel disease (IBD), including Crohn's disease and ulcerative colitis, Insulin Dependent Diabetes Mellitus (IDDM), Interstitial lung disease, juvenile diabetes, Juvenile Arthritis, juvenile idiopathic arthritis (JIA), Kawasaki's Disease, Lambert-Eaton Myasthenic Syndrome, Lichen Planus, lupus, Lupus Nephritis, Lymphoscytic Lypophisitis, Ménière's Disease, Miller Fish Syndrome/acute disseminated encephalomyeloradiculopathy, Mixed Connective Tissue Disease, Multiple Sclerosis (MS), muscular rheumatism, Myalgic encephalomyelitis (ME), Myasthenia Gravis, Ocular Inflammation, Pemphigus Foliaceus, Pemphigus Vulgaris, Pernicious Anaemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune cholangiopathy, Psoriasis, Psoriatic arthritis, Raynaud's Phenomenon, Reiter's Syndrome/Reactive arthritis, Restenosis, Rheumatic Fever, rheumatic disease, Rheumatoid Arthritis, Sarcoidosis, Schmidt's syndrome, Scleroderma, Sjörgen's Syndrome, Stiff-Man Syndrome, Systemic Lupus Erythematosus (SLE), systemic scleroderma, Takayasu Arteritis, Temporal Arteritis/Giant Cell Arteritis, Thyroiditis, Type 1 diabetes, Type 2 diabetes, Ulcerative colitis, Uveitis, Vasculitis, Vitiligo, and Wegener's Granulomatosis.

12. The method of claim 10 wherein the autoimmune inflammatory disease is systemic lupus erythematosus.

13. A method of treating an antibody-mediated disease in a subject in need thereof, comprising administering to the subject a therapeutically effective amount of at least one compound selected from the group consisting of an HIV-protease inhibitor, diflunisal, anethole, rosiglitazone and daunorubicin.

14. The method of claim 5 wherein the cancer is a B-cell tumor.

15. The method of claim 10, wherein the step of administering reduces CD95-mediated lymphocyte motility and/or B cell maturation in the subject.

16. The method of claim 13, wherein the antibody-mediated disease is graft rejection, graft vs. host disease, or an inflammatory-autoimmune disease.

17. The method of claim 14, wherein the B-cell tumor is a multiple myeloma tumor or a chronic lymphocytic leukemia tumor.
